(19) United States
(12) Patent Application Publication
(10) Pub. No.: US 2022/0104883 A1
(43) Pub. Date: Apr. 7, 2022

(54) POSITION SENSING SYSTEM FOR MEDICAL DEVICES, ORTHOPEDIC DRILL OR DRIVER, AND METHOD OF PERFORMING SURGERY

(71) Applicant: EXTREMITY DEVELOPMENT COMPANY, LLC, Akron, OH (US)

(72) Inventors: David B. KAY, Akron, OH (US); Bryan DEN HARTOG, St. Paul, MN (US); Dustin DUCHARME, Littleton, CO (US); Robert A. CHARLES, New Boston, NH (US); Gregory HURLEY, Windham, NH (US); James J. KENNEDY, III, Mont Vernon, NH (US); Richard M. THOMAS, Bow, NH (US); James M. PESCHKE, Croydon, NH (US); Aaron MONCUR, Mesa, AZ (US); Ian P. KAY, Fairlawn, OH (US); Quang-Viet NGUYEN, Aldie, VA (US); Jon TAYLOR, Groton, MA (US)

(21) Appl. No.: 17/426,462

(22) PCT Filed: Jan. 29, 2020
(86) PCT No.: PCT/US2020/015637

§ 371 Date: Jul. 28, 2021

Related U.S. Application Data
(60) Provisional application No. 62/798,751, filed on Jan. 30, 2019.

(51) Int. Cl. A61B 34/20; A61B 17/17; A61B 34/30; G16H 40/63

(52) U.S. Cl. CPC A61B 34/20; A61B 17/1703; A61B 34/30; A61B 2090/3983; A61B 2034/2063; A61B 2034/2057; A61B 2034/2065; G16H 40/63

(57) ABSTRACT

A medical device en-vivo positional determination system using a plurality of ultrasonic transducers for the time-of-flight (TOF) determination of absolute linear and angular positional information of the tool bit tip for the purposes of more accurate hand-held drilling, cutting, etc. on the work piece using digital code modulation schemes and digital signal processing (DSP) to provide a real-time display of the 3-linear position and 2-angular orientation of the tool bit (drill, scalpel).

### FIELD OF THE INVENTION

The field of this invention is in the area of medical devices, and more specifically, medical devices used by qualified personnel such as physicians and nurse practitioners, (and most notably surgeons of various specialties including orthopedic generalists, orthopedic and podiatric extremity specialists, spinal surgeons and neurosurgeons) during medical procedures, and especially surgical procedures. More specifically, this invention is related to relatively small and cost efficient hand-held surgical devices, such as a drill or wire driver, and tools or apparatus which can be sterilized, or which have a cost structure that would permit single use so that they are “disposable”, and to methods of surgery that incorporates such devices.

### BACKGROUND OF THE INVENTION

While there has been a substantial body of work and commercial products which provide imaging assistance or robotic guidance, (i.e., “surgical navigation”) during surgery, the devices have been “large box” devices for example million-dollar devices owned and leased to the practitioner by a hospital or healthcare institution, and that are lodged in dedicated surgical environments. These devices require a very large capital investment, which includes the cost of the surgery room and environmental controls, training for dedicated personal, and an expensive and complex device. Moreover, these devices tend to be large and invasive in the surgery and may even dictate the surgical environment such as the space and temperature requirements around these devices.

Since these “big box” devices include complicated hardware and software and very high development costs, there has been very little development with respect to lower cost hand-held surgical devices with positional feedback for medical use since these devices have limited cost elasticity, and uncertain return on the development and production costs, in addition to cost absorption, payment or reimbursement issues. Thus, typical “targeting” is presently limited to the hand-eye coordination of the practitioner performing the procedure. As discussed herein “targeting” refers to the guidance in time and through space of the trajectory and depth of an instrument workpiece within a biological environment, which typically involves highly sensitive areas and highly critical positioning and time constraints. Depending on the medical specialty or even the area of the body being treated, the “workpath” may have constraints that include the start point, the end point, and the path between, especially for areas with high concentrations of sensitive and functional or life threatening implications, such as the spine, extremities, the heart or the brain or areas critically close to nerves, arteries or veins.

For procedures in which the precision of the cutting or drilling of a target pathway located within a physical patient body is crucial (i.e., the “workpath”), the skill and hand-eye coordination of the surgeon is of paramount importance. Due to the nature of hand-held tools, and the dynamic and flexible nature of the “work area” within a patient body, errors of the tool tip versus ideal positioning during use can, and will, occur regardless of the skill of the working practitioner. This possibility is increased with user fatigue that can be physical and mental in origin, as well, as issues relating to inexperience, and differing surgical conditions, such as bone or soft tissue quality.

It is the aim of the present invention to reduce these errors by providing the surgeon with a real-time indication of the “workpath” of the tool relative to the anatomical site. In certain types of surgery, real-time radiography using x-rays provides the surgeon with the knowledge of positional information that would otherwise be invisible due to the opaqueness of the site. However, this is not always possible, and certainly, it is not desirable to use radiography in real-time as the exposure to x-rays can be considerable for both the patient and the surgeon. Thus, it is desired that the position of the tool tip relative to a desired “workpath” be provided by a means that minimizes any health risk as a result of the surgery to the patient or surgeon

### SUMMARY OF THE INVENTION

The present invention addresses the need for a device which is distinguished from the prior art high capital “big box” systems costing hundreds of thousands of dollars and up. This invention further relates to a method for the accurate real-time positional determination in three dimensions of a surgical instrument workpiece relative to the end point or pathway within the patient body (i.e., the “optimal course” or “workpath” of the instrument workpiece) in the operating room, for procedures including, among other things, drilling, cutting, boring, planning, sculpting, milling, debridement, where the accurate positioning of the tool workpiece during use minimizes errors by providing real-time positional feedback information during surgery and, in particular, to the surgeon performing the procedure, including in an embodiment in line of sight, or in ways that are ergonomically, advantageous to the practitioner performing the procedure.

In a narrow recitation of the invention, it relates to a guidance aid for use by orthopedic surgeons and neurosurgeons that is attached to a standard bone drill or driver and operates so as to provide visual feedback to the surgeon about how close the invasive pathway is during the drilling operation to an intended orientation and trajectory. Thus, the invention permits the surgeon to use the visual feedback to make course corrections to stay on track, and as necessary to correct the trajectory of a workpiece. In the past, surgeons would use a mechanical “jig” to help guide the position of the intended starting point, and the end point of a drill pathway (i.e., the drill hole), but the present invention uses electronic, and preferably ultrasonic, senders and receivers borne by a hand-held instrument with a visual display and feed-back system to inform the surgeon as to how to create a drill pathway through a subject patient body part which is contained within a three-dimensional reference frame. By “hand-held”, it is meant an instrument that weighs under five pounds and has a configuration that allows it to be manipulated in the hand of a user. Reference points are obtained through digital images, for example, captured using fluoroscopy.

The system of the invention establishes a frame of reference for the anatomical subject area to allow a user to mark reference points through the placement of markers (i.e., fiducials) to define a top and side plane, and an independent imaging system is used to visualize the anatomical site, while the system includes means to determine, and mark starting and end points relative to the anatomical subject area and input them into the reference system. The guidance system works within the marked reference area to determine the location of sensors, preferably ultrasonic receivers or senders, carried on the hand-held instrument.

Thus, the invention relates to a surgical targeting system guided by ultrasonic sender/receiver pairs that are strategically mounted on the hand-held (or potentially robotic) drill. The sender/receiver pairs are in proximity to x-ray opaque fiducials which are positioned relative to the subject surgical area (i.e., the anatomy of the patient which is located within a defined three-dimensional reference frame) and which determine the proximity in space of the associated ultrasonic sender/receivers as they change course over time (i.e., by calculating the “time of flight” or TOF of the generated soundwaves).

The markers and the drill entry and end points are selected by the user (surgeon) and entered into a computer program residing on a CPU member that accesses software to display or represent the drill pathway of the surgical workpiece in the subject surgical area on a GUI (“graphical user interface”) as determined by the relationship between the ultrasonic sender/receiver pair(s) with the reference frame of the system. Thus, the system allows the display to inform the user as to the trajectory of the instrument and the depth of penetration into the anatomical site which can be displayed in a number of ways, including reticles or cross-hairs, circle in circle, numbers, colors or other alignment methods including in separate visuals or combined.

In accordance with the present invention a plurality of ultrasonic transducers acting as sound pulse transmitters are mounted on a reference frame that is represented by a base plate which is positionally fixed relative to the surgical site (i.e., the physical environment within or about the patient's body). In this case, the surgical site may also need to be positionally fixed or restrained within the reference frame. A plurality of ultrasonic transducers acting as sound receivers (microphones) are mounted on the tool handle. An electronic microprocessor system synthesizes the sound pulses which are generated by the transmitter transducers, and digitizes the received sound pulses and performs the necessary algorithms such as FFTs, correlation functions, and other digital signal processing (DSP) based algorithms performed in hardware/software, thus provides the real-time positional information for the surgeon for example, via an electronic screen such as in “line of sight” on the tool handle itself or on a separate monitor, including a display that could be linked to the system, such as on a head's up display screen worn by the surgeon or a dedicated display that is located at a position that is ergonomically advantageous for the user. The tool can be any tool used by a medical practitioner, including for example, a scalpel, saw, wire driver, drill, laser, arthroscope, among others.

In the simplest embodiment of this invention, the tool handle will support and/or house a plurality of the ultrasonic receivers mounted in an orthogonal fashion such that 6 degree of freedom (DOF) information regarding the linear (x, y, z) position, and the angular (yaw, pitch, roll) can be obtained from the knowledge of the vector positions. At a minimum there are 3 ultrasonic receivers via the TOF (Time of Flight) of the ultrasonic pulses from the transmitters to the receivers, but preferably 4 ultrasonic receivers to provide redundancy.

By means of the targeting assistance provided by the present invention, it is further desired that 5 degrees of freedom (DOF) positional information be provided in real-time at rates of up to 5 Hz, preferably 10 Hz and most preferably up to 15 Hz or even 30 Hz, with a positional accuracy of +/−3 mm, preferably 2 mm, and most preferably 1 mm, in 2 or 3 linear dimensions, and angular accuracy of +/−3° and preferably 2° in 2 angular dimensions of pitch and yaw, and that this positional information be obtainable in a 0.75 m×0.75 m×0.75 m, and preferably 0.5 m×0.5 m×0.5 m cubic working volume.

In the present invention, a plurality of ultrasonic transducers (i.e., at least 3 and more precisely from 3 to 15, or 3 to 10 where the excess from a three-dimensional matrix are used for an array) are used to provide the positional information of a tool relative to a plurality of microphones or ultrasonic receivers supported or mounted relative to or on the tool. The distances from the transmitters to the receivers are calculated by a time-of-flight (TOF) propagation of the transmitted sound pulse. With the use of the local speed of sound at a given temperature, a distance from the transducer to the receiver can be calculated. The use of phase extraction from the FFT provides some immunity to amplitude noise as the carrier frequency is at 20-75 kHz, and preferably 40 kHz+/−5 kHz. The use of certain coding schemes superimposed upon the carrier frequency permits the increase in signal to noise ratio (SNR) for increased immunity to ambient noise sources. Other means of extracting distance or positional information from ultrasonic transducers for robotic navigation have been described by Medina et al. [2013], where they teach that via use of a wireless radio frequency (RF), coupled with ultrasonic time-of-flight transducers, positional information with up to 2 mm accuracy can be obtained in a space as large as 6 m for tracking elder movement. Segers et al. [2014, 2015] has shown that ultrasonic pulses can be encoded with frequency hopping spread spectrum (FHSS), direct sequence spread spectrum, or frequency shift keying (FSK) to affect the determination of positions with accuracies of several centimeters within a 10 m space. More recently, Khyam et al. [2017] has shown that orthogonal chirp-based modulation of ultrasonic pulses can provide up to 5 mm accuracy in a 1 m space. However, none of these previous studies have been able to provide a 2 or 1 mm accuracy for a system that fits within an operational size space that is the size of the intimate volume direct affected by most medical procedures (i.e., about 1 cubic meter or less), which is the goal of the present invention.

In a more advanced embodiment, the tool and the base for the work piece will also contain visual fiducial markers that will assist a double set of video cameras mounted orthogonally as to produce a top view and a side view so that the fiducial markers can be used with video image processing to deduce spatial information that can be used in conjunction with the ultrasonic positional information, and in particular to set target locations rather than as an adjunct to determine drill position in real time. Thus, the system of the invention allows the use of x-ray imaging for testing purposes so as to eliminate unnecessary exposure to users to radiation during surgery.

And in yet a further advanced embodiment, the digital signal processing (DSP) of the ultrasonic signals will utilize phase-inversion detection of an audio signal encoded with a high-contrast code so that the TOF information can readily be detected from the background noise, and also so that a plurality of transducers can be encoded with different coding schemes to provide an orthogonal basis set of acoustic signals for the accurate positional determination of a solid object with 5 degrees of freedom (DOF) position information. Such codes include the “AA55” coding scheme where an even number of 4 to 12, and preferably 8+/−2 sets of pulses are generated with the first three to six, and preferably four with 0 degree and last same number, i.e., four, with 180 degree phase offsets. Certain types of coding schemes have been shown to demonstrate higher signal to noise ratio (SNR) than others.

In a third embodiment, the ultrasonic transducer system above is used in conjunction with a fluoroscopic radiography system to provide both contextual imaging, coupled with quantitative positional information for the most critical types of surgery (which can include spinal surgery, invasive and non-invasive neuro surgery or cardiac surgery, for example). Thus, the invention also relates to methods of performing medical procedures including surgery and dentistry that establishes a frame of reference for the anatomical site, and wherein a medical tool supports sensors to locate and guide a medical procedure on the anatomical site within the frame of reference. As an example, the present invention relates to a procedure involving a guided procedure to percutaneously implant guide wires in a femoral neck for a non-invasive cannulated screw fixation of a hip fracture.

All of the above embodiments allow for the real-time display of the absolute positional information of the tool work piece and preferably the tool tip, relative to the body part, intended target position, and the desired “workpath”. The display could show a delta distance reading relative to the intended target position so that the surgeon is simply looking to minimize the displayed delta numbers or a graphical or other visual representation thereof (e.g., circle in circle). The display can show the x, y, z positions to the nearest millimeter or partial millimeter and also the yaw and pitch to the nearest degree or partial degree, including the incremental changes of these values. The angle of approach is often an important parameter for certain procedures such as a wire drill and especially where the start point may be known, and the end point maybe marginally understood, but the path between may only have certain criteria.

It is also the aim of this invention to provide this positional information in a lightweight tool handle that is unobtrusive and easy to use, and as similar to the existing instrument as possible, such that the transition to use of the system of the invention is user friendly and seamless to the practitioner. It is a further goal of this invention to have a tool handle and base plate with transmitters that are easy to sterilize, including by autoclave, or which are cost-effective enough for manufacture in whole or in part, as a disposable one-time use system.

It Is one advantage of the present invention that it can be very compact and unobtrusive by nature of the form factor, and the possibility of being wireless, and the positional sensing is affected by sound waves compared to mechanical position sensors such as articulated multi-joint angular-feedback linkages, and further that the invention can be safely used in a healthcare facility without hindrance by external noise or without contaminating other wave uses in the facility.

Another advantage of the present invention is that it permits the surgeon to manually hold the tool in a natural manner that does not have any mechanical resistance, such as that might be encountered with as articulated multi-joint angular-feedback linkages, and with a footprint and size that can be easily manipulated and which is similar so much as possible to the tools that they are already comfortable using.

It is another advantage of the present invention that it can provide both position and angular information simultaneously, and advantageously, sufficiently in ‘real-time’ to enable the use during surgery.

It is another advantage of the present invention that it potentially has increased immunity over typical ambient background noise sources since it works in the ultrasonic frequency band, and the data processing occurs via FFT in the frequency domain where typical mechanical and ambient noise source or interference amplitudes are minimized through the 1/f principle where noise amplitude is inversely proportional to the noise frequency.

It is another advantage of the present invention that it can be used to augment radiography techniques such as fluoroscopy or x-rays to provide an additional level of information that is quantitative and can be used for the “last inch” deployment of a surgical tool for critical procedures where accuracy is of paramount importance.

It is another advantage of the present invention that it provides the surgeon with positional sensing system that is absolute relative to the working base reference system and is free from dead-reckoning (propagation-based) errors that are inherent in some other types of (non-absolute) positional sensing.

It is an additional advantage of the system that it serves as a three-dimensional aiming system that as a single use or low cost hand-held instrument includes a system that helps the user (a surgeon or robot) determine the work angle for a work piece integral to the instrument from an identified point of entry in an anatomical work area to a desired end and provides feed-back by display or tactile means to correct the alignment of the work piece to achieve and/or maintain the desired alignment. The system can be used in surgery, or for training purposes (including for example using proprioceptive corrections to, alert a user to alignment issues, such as is used in a haptic setting) with an instrument, such as a drill or wire driver or for the implantation of implants including pegs, nails and screws. Examples of suitable surgical method using the present invention include hip fracture fixation where a screw or nail is inserted into the greater trochanter using the present targeting, aiming or guidance system or instrument, or for use in hammer toe fixation which can include phalangeal intramedullary implants.

### BRIEF DESCRIPTION OF THE DRAWINGS

FIG. 01 shows a schematic diagram of the preferred embodiment of the present invention;

FIG. 02 shows a schematic diagram of the principle of operation;

FIG. 03 shows an alternate embodiment which includes two digital video cameras;

FIG. 04 shows a block diagram of the steps and sequence used to acquire and derive the distances and angles from the video imagery and ultrasonic audio signals generated and collected;

FIG. 05 shows a digitized oscilloscope trace of the AA55 coded signal and the received signal from the microphone;

FIG. 06 shows the ultrasonically determined distance vs. true distance for the range 492 mm to 532 mm (prior to offset calibration);

FIG. 07 shows the ultrasonically determined distance vs. true distance for the range 510 mm to 514 mm (prior to offset calibration);

FIG. 08 shows the ultrasonically determined distance vs. true distance for the range 638 mm to 642 mm (prior to offset calibration);

FIG. 09 shows an alternate embodiment of FIG. 01 wherein the transducers are mounted at the back of the tool handle to provide more clearance around the tool distal end of the tool bit for working in tighter areas;

FIG. 10 shows the perspective view of the base plate, cameras, transducers, fiducial markers, and tool handled instrumented with the receivers and display screen and microprocessor for the present invention;

FIG. 11 shows a side view of FIG. 10 for clarity;

FIG. 12 shows a side view of the present invention, superimposed with 3 conic sections representing the acoustic beams of the transducers and their intersection, relative to the rest of the mechanical parts of the overall system;

FIG. 13 shows a perspective view of FIG. 12 for clarity, and it is evident that the acoustic beams for Transducers 1, 2, 3 are represented by cones of propagation, the 3D intersection of these 3 cones is the “active” region of the present invention where the positional information can be determined unambiguously;

FIG. 14 shows a Top View of FIG. 13 for added clarity, and depicts the angular zone of coverage that the present invention provides based on the intersection of the 3D cones represented in FIG. 13;

FIG. 15 shows a photograph of the Display Screen as seen by the Operator, wherein the live (near real-time) X, Y delta-positions and delta-distance-to-target plus a Cross-hair reticle with live target are also shown to provide the operator with position and angular approach tool-path information;

FIG. 16 shows an x-ray of a typical procedure where the present invention would be used to help guide a wire drill for hip fixation; and

FIG. 17 shows a second x-ray of the typical procedure where the present invention would be used for hip fixation.

### DETAILED DESCRIPTION OF THE INVENTION

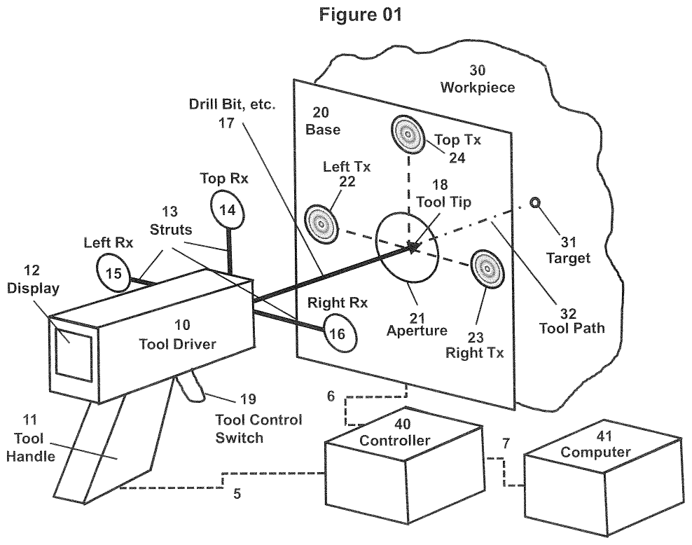

In the preferred embodiment of the present invention as shown by the schematic diagram in FIG. 01, a tool driver 10, with handle 11, a visual display screen of the measured position information 12 is provided. The tool driver 10 is also fitted with struts (supporting rods) 13 that serve to hold at least three receiver microphones or transducers (microphones) at the top 14, left 15, and right 16 positions. The tool driver has a tool bit (k wire, drill, scalpel, etc.) 17, which has a distal tip 18 which corresponds to the spatial positional information shown in the display 12. The transducer receivers 14,15, 16 (e.g., Murata or Knowles wideband MEMS analog ultrasonic microphone) are in acoustic communication with their respective acoustic transmitters (piezo ultrasonic transmitters), top 24, left 22, and right 23, respectively. These acoustic transmitters are secured to a rigid base plate 20 that serves to locate the transmitters with respect to the work path in the surgical environment in the patients body part 30 subject to the procedure, to guide the tool tip 18 through an aperture 21 in the base 20, along the workpath 32, towards the target 31. The acoustic transmitters 22, 23, and 24 are in direct or indirect electrical communication with an electronic controller unit 40 via wiring cable 6 or by electronic transmission, such as Bluetooth. The controller 40 is also in electrical communication with the tool driver 10, and in communication, at least during imaging, with a computer 41, a via means such as wire cabling 5 and 7, respectively. Together, these components shown in FIG. 01 form the basis of the present invention's preferred embodiment that utilizes the measurement of the TOF (“Time of Flight”) of a sound pulse from the transmitters 24, 22, and 23, to the receivers 14, 15, and 16, respectively. By use of geometrical relationships, the fixed distances between the individual receivers and transmitters, and the speed of sound, the precise distances between the spatially separated transmitters and receivers can be determined with a closed form equation calculated either in the controller unit 40, the computer 41, or even through use of a microcontroller in the tool driver 10 itself and then displayed on the screen 12. In this sense, the system can be predictive of the continued course of the tool-tip along the workpath, although, it should be understood that the system tracks the position and displays it in near-real time during use.

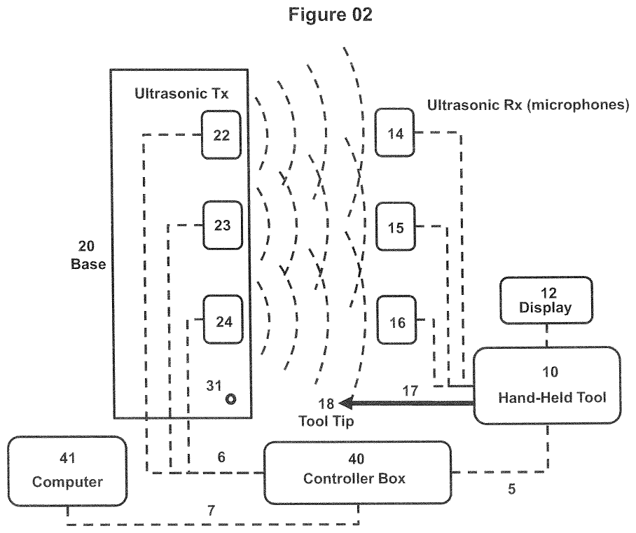

FIG. 02 schematically illustrates the principle of operation of the present invention. The acoustic transmitters 22, 23, and 24 are shown attached to rigid support base 20 and in electrical communication via a wiring cable 6 to a controller box 40, while also being in acoustic communication with acoustic receivers 14, 15, and 16. The acoustic transmitters send an audio signal consisting of a series of pulses at an ultrasonic frequency (circa 40 kHz+/−10 kHz) which are then received by the receivers located on the tool driver 10, and then sent to a controller box 40 via electrical wire cable 5, where the signal is processed. As a specific example the transmitter emits pings consisting of either cycles at 40 kHz where each “ping” contains four cycles at zero degrees (relative to start) followed by four cycles at 180 degrees phase. Each transmitter emits the same pulse train in succession. The emitted pulses are spaced so as to prevent overlap of successive transmissions by any transmitter. Once a pulse is emitted the on-board analog-to-digital-converters (ADC) begins sampling at a rate of 2 mega-samples per second (MS/s) into a buffer. The contents of these three buffers are used to compute the distance between the operating transmitter and each of the three receivers. There is a fixed delay between the start of the transmission and the start of the ADC reception. This delay is constant and is added to the time calculated by the distance computation. The processing can also be performed in a computer 41, connected to the controller box 40 by electrical wire cables 7. By calculating the time delay between the transmitted acoustic pulses 32, and when they are received. This time delay or TOF and the speed of sound can then be used to calculate the distance corresponding to the TOF. The calculation of the TOF can be effected through various methods using digital signal processing (DSP) within either the controller box 40, the computer 41, or even the microcontroller in the hand-held tool driver 10. It is noted that the computer 41, and the microcontroller 10, can be the same component or two separate components.

A time-of-flight (TOF) propagation of the transmitted sound pulse can be used in the calculation of the distances from the transmitters to the receivers. In particular, this calculation can include phase information from the Fast Fourier transform (FFT) of the sound waves emitted from the transmitter(s) onto the receiver(s), which is proportional to the time delay of the transmitted pulse to the received sound pulse. DSP algorithms that can be applied to extract the time delay to get the TOF, include: the Fast Fourier Transform (FFT), the convolution of the transmitted and received pulses, or threshold algorithms looking for phase inflection of coded pulses. Such a phase inflection can be obtained by modulating the 40 kHz carrier frequency with coded sequence of 0 deg and 180 deg phase bits. Development work was performed in order to locate the phase inflexion point using strictly algorithmic means (i.e., without human intervention). During Laboratory testing, Octave code which reads in analog data taken from an oscilloscope capture produced the final measurement output. Octave outputs a “Sample Number’ corresponding to its detection of the start of the final audio pulse prior to phase inversion. This is usually, but not necessarily, the pulse with the highest peak amplitude. This number, divided by the sample rate of the oscilloscope (3.125 MS/s) and multiplied by the speed of sound gives the preliminary ultrasonic distance measurement. For the prototype the Octave code algorithm and oscilloscope measurements were replaced by signal processing and ADC measurements of an embedded microcontroller

For example, the AA55 code is given by four consecutive 0 deg pulses followed by four 180 deg pulses. Once the coded signals are received and digitized, they can be processed with a DSP algorithm that looks for the change from 0 deg to 180 deg, which provides a high contrast signal on top of a noisy background. The algorithm also takes into account the physical dimensions and locations of the acoustic transmitters 22, 23, and 24, and the acoustic receivers 14, 15, and 16, relative to their mechanically defined supporting structures consisting of the base 20, or the hand-held tool driver 10, along with the physical dimension of the tool bit length, and location of the target 31.

The TOF calculation can use a correlation method for detection of the beginning of a waveform, or a phase detection method, which uses an inflection point of a phase change and may have to account for sources of off-set, such as the wave number, microphone displacement, or transmitter/receiver foci. In turn the offset may need to account for variables, such as temperature and ambient environment.

The CPU uses an algorithm that takes a known reference waveform based on actual data and performs a correlation to the received data. Specifically, the system uses an Octave language program “MAKECFile” routine to create a source file containing the reference waveform used by the embedded software. The following steps are followed for each receiver: 1) Find the peak amplitude of the received waveform; 2) Go back one reference waveform image length from the peak amplitude (currently 512 samples); 3) Correlate the entire waveform to the received reference waveform using a starting window from PEAK-512 to PEAK and stepping through the correlation in 500 nanosecond (ns) steps, until the window reaches PEAK to PEAK+512; and 5) The time where this correlation is the highest is the beginning of the received wave front.

This correlation is performed using a discrete Fast Fourier Transform technique, which has a faster execution time than a straight correlation. The FFT correlation algorithm and the conventional correlation algorithm are detailed in the discrete Fourier transform correlation (DFTCorr”) code.

The previous process produces three integer values, each indicating the number of analog-to-digital-converter (ADC) sampling count times (in 500 nS steps) after each transmission when each receiver “hears” the incoming waveform.

For the prototype 4 transmitters and 3 receivers are used. In the general case, this can be expressed as “n” transmitters and “m” receivers, then the number of distance calculations is “n×m”. Once all transmitters have “pinged” their sounds, the distance algorithm will have n×m (i.e., 12) integers corresponding to the distances between the n, where n is 3-10, and preferably 3-6 and specifically four transmitters and m, where m is 3-10, and preferably 3-6 and specifically three receivers. The integers are converted (using speed of sound in air, and at a known temperature) into distances in millimeters. The speed of sound is proportional to the square root of the absolute temperature and is approximately 342 m/s at ambient conditions. Each 500 nS measurement interval represents 0.17 mm, for the example, the speed of sound, of distance precision. The next step is to compute the location of each transmitter in the coordinate space of the three receivers. To ease in trilateration, the receiver coordinate system is defined placing Receiver #1 on the origin (0,0,0), Receiver #2 on the x-axis (125,0,00 and Receiver #3 in the z-plane (62.5,−108.25,0) for the example frame geometry used in the prototype. Trilateration of each of the four transmitters is performed using trilateration coordination computer using the equations:

$$= \frac{\begin{matrix}2 \\ 1\end{matrix} - \begin{matrix}2 \\ 2\end{matrix} + \begin{matrix}2 \\ \end{matrix}}{2} \\ = \frac{\begin{matrix}2 \\ 1\end{matrix} - \begin{matrix}2 \\ 3\end{matrix} + \begin{matrix}2 \\ \end{matrix} + \begin{matrix}2 \\ \end{matrix}}{2} -- \\ = \pm \sqrt{\begin{matrix}2 \\ 1\end{matrix} - \begin{matrix}2 \\ \end{matrix} - \begin{matrix}2 \\ \end{matrix}}$$

Where r₁, r₂, and r₃ are measure distances between the transmitters and receivers 1, 2, and 3 respectively, d is the X coordinate of Receiver #2, 1 is the X coordinate of Receiver #3, and j is the Y coordinate of Receiver #3. Trilateration can, in the general case, produce unsolvable results if the root of computing Z becomes negative. If this happens in practice, the result is not used, however, the algorithm normally computes a solvable result.

Since the geometry of the transmitters and the relative location of the target is known, the location of the target may be determined once the location of the transmitters is known. Also, it is possible to calculate the true speed of sound from the measured distances compared to the known geometry of the transmitter and receiver arrays and thus compensate for changes due to temperature during the measurement process. Since trilaterated transmitter locations will never exactly align with the reference locations, a means is required to resolve these inaccuracies. This can also be solved by trilateration. At start-up, the algorithm computes and stores the distance between the target and each transmitter or this information is known from the prior imaging measurements. These distances are used at runtime to trilaterate the location of the target once the transmitter positions are known. “Mapper” software is used to detail this process. Since only three distances are needed to perform a trilateration, four transmitters can ideally produce four answers for the target location. At present, all valid answers are simply averaged to produce the final target position. Once the coordinates of the target are known in receiver coordinate space, a final translation plots this position relative to the drill tip.

Code modules and algorithms described above were first developed using the Octave language and C code to run on a PC for development and test. The DFTCorr and Mapper codes were used for development and the operational code was ported to firmware using a development environment for the ARM processor on a STM Nucleo-F767Zi development board. The hardware abstraction layer (HAL) and start-up code was generated using the STMCubeMx software suite and making use of the STM32F7 locater”.ioc” file.

For the ping, a serial peripheral interconnect (SPI) port is conFigured to provide the programmable pulse trains to drive each transmitter. The MCU is conFigured to perform simultaneous conversion of three ADC channels (one for each receiver) at 2 MS/s. Each ADC value is transferred via direct memory access (DMA) to a sequential buffer. The binary data is sent to the SPI port in order to generate the desired output waveform and each of the four pingers is excited individually while the ADC readings are stored in a buffer. When all of the transmit (Tx) transducers (a.k.a. “pingers”) have been excited, the ADC data buffers are processed. A software adjustable gain IC is used to keep the receiver output level within a useable range. The ADC data is processed using the ARM library FFT algorithm to correlate the output waveform to an ideal waveform to locate the beginning of the response to the generated wave. The beginning of the wave is identified in the form of number of ADC samples from the start of sampling. This number is converted into milliseconds of delay using the ADC sample frequency. Using the calculated delay and an estimated speed of sound, the distance from each pinger to each receiver is calculated. Trilateration is used with the known distances to calculate the orientation of the drill. The display code used graphics display libraries provided by the STMCubeMx development environment. The display is updated after a complete set of pings is processed. The display shows the error in orientation between the target and the drill using a fixed and movable set of crosshairs. Redundant information from the four transmitters can be handled by averaging, or worst in or most mover out, and the algorithm could be optimized to minimize bounce between successive pings. In addition, filtration methods, such as Kalman filtering can be applied to the final location to smooth out infrequent correlation distance errors.

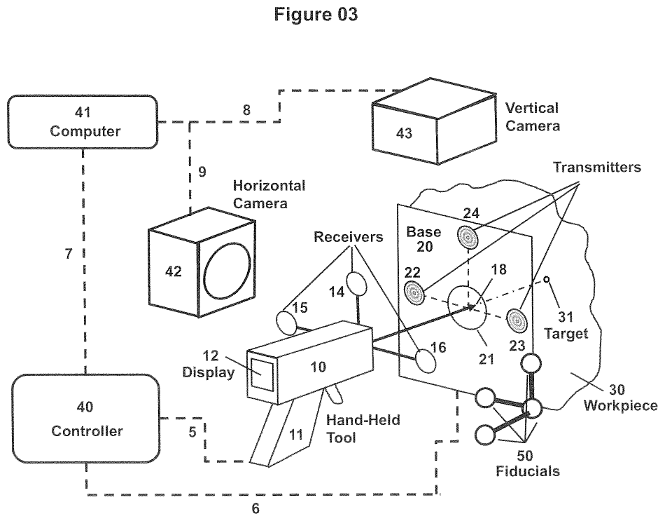

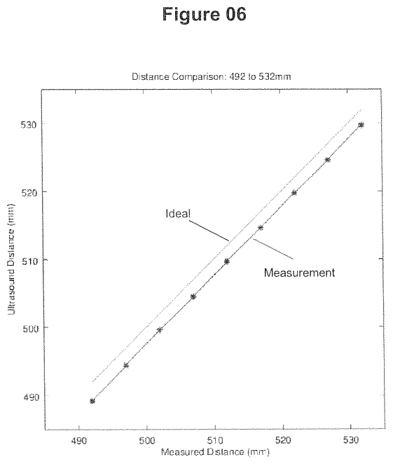

Tests were conducted by varying the transmitter/receiver distances while recording data at 0.5 mm increments. Example measurements are shown in FIGS. 06,07,08. from 1) 492 mm to 532 mm; 2) 510 mm to 514 mm; 3) 382 mm to 386 mm; and 4) 638 mm to 642 mm. The correlation for plotting the ultrasound distance to the measured distance was close to ideal with a fixed offset that calibrates out. FIG. 03 shows a schematic diagram of an alternate embodiment that has the same components as described in FIG. 01, but now has additionally, two orthogonally positioned digital video cameras that view the hand tool vertically from above 43, and horizontally from the side 42, along with the tool handle 10 which uses the receivers tool bit tip 18 as fiducial markers for the tool handle 10, along with visual spatially-fixed fiducial markers connected to the base plate 20, to provide a visual reference so that digital image processing via the computer 41, can also be used in addition to the positional information obtained from the acoustic transmitters 22, 23, and 24 and the receivers, 14, 15, and 16. In FIG. 03, the digital video cameras can also be substituted with radiographic cameras to observe x-rays transmitted through the work piece 30 as in fluoroscopy.

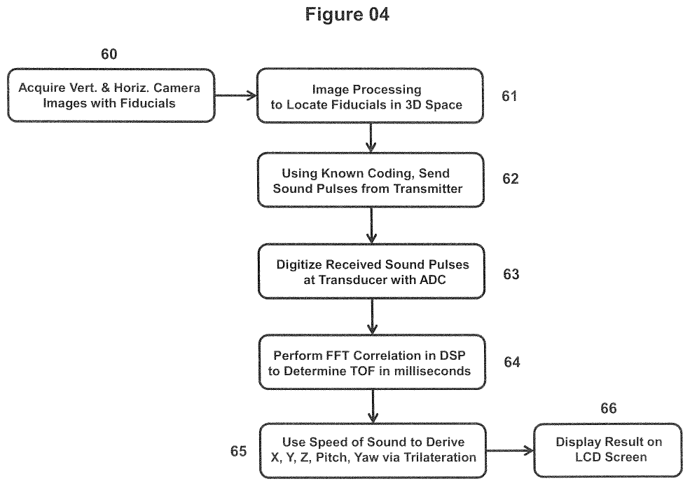

FIG. 04 shows a block diagram of the method of deriving the spatial measurement using the system depicted in FIG. 02. In the first Step 60, the vertical and horizontal video cameras acquire an image containing the fiducial markers to establish and locate the target. The target location is thus known relative to the fiducials. The fiducials and transmitters are part of a fixed geometry frame. Since the 3D distances from the fiducials-to-transmitters are known, the target-to-transmitter distances are now known. Using a known coding scheme such as the AA55, a series of coded pulses are synthesized and sent to the transmitter transducers where an ultrasonic pulse is generated as shown in Step 62. The coded ultrasonic audio signal then propagates through free space with a certain TOF whereupon it is received and converted to an electrical signal by a microphone and then digitized with an ADC in Step 63. The digitized signal is then processed via DSP using a discrete Fourier transform correlation and the time-step of the correlation is detected and measured in Step 64. By using mechanically fixed dimensions of the transducers, the length of the tool bit, the speed of sound, and other factors, the 5 DOF positional information is calculated using geometric relations via trilateration. The resulting information of the 5 DOF spatial position is then displayed on a display screen as shown in Step 66.

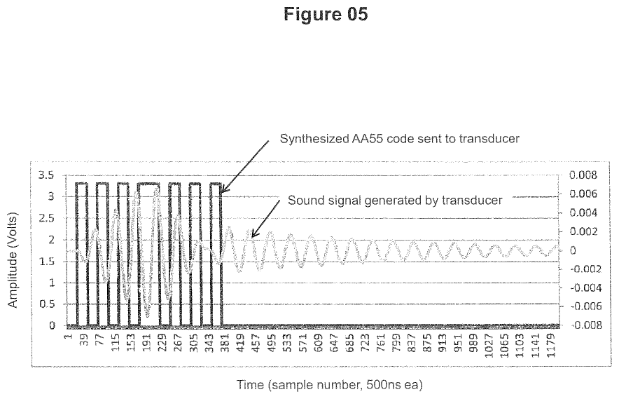

FIG. 05 shows the digitized signal from the oscilloscope which was used to capture and display the electrical signal used to drive the ultrasonic transmitters with an AA55 code (dark trace), and the resulting measured electrical signal from the receiver microphone (lighter) with zero time delay as shown here. The highest peak signal represents the 5th cycle corresponding to the phase inflection point.

FIG. 06 shows a comparison of the ultrasonically measured distance (vertical axis) versus the actual measured distance (horizontal axis) over a measured range from 490 mm to 535 mm, using a wideband microphone element as the receiver and the AA55 coding scheme with an automated processing of the TOF using the DFTCorr algorithm in the DSP. The ideal variation is the solid line without points. As can be seen, the ultrasonically measured distance is highly linear but has a slight offset from the ideal value. This offset may be the result of a difference in the speed of sound at different temperatures or mechanical tolerances in mounting of transmitters or receivers and can be easily calibrated out with a single point calibration to remove the offset.

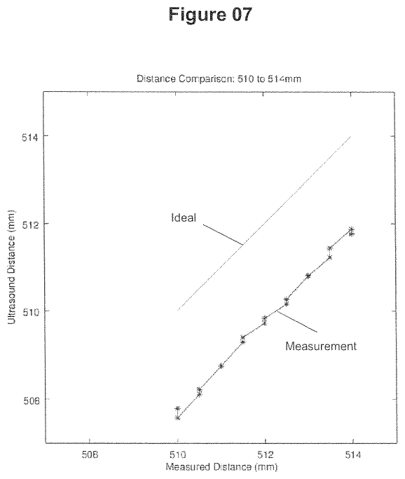

FIG. 07 shows a similar measurement to that shown in the previous Figure, except over the measured range of 510 mm to 514 mm. Of note in FIG. 06,07,08 is the variation in the ultrasonically measured distances versus the true distance was less than 1 mm once the offset due to temperature calibration is removed.

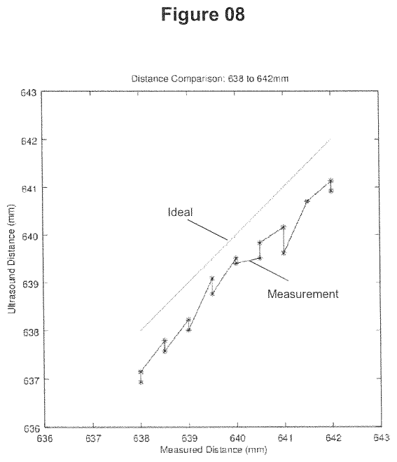

FIG. 08 shows a similar measurement to that shown in the previous two Figures, except that the range of actual distances measured was from 638 mm to 642 mm. Again, we see that the ultrasonically measured distances are very linear, but there is an offset due to temperature or mechanical tolerances, that can easily be calibrated-out with a single point calibration. Also shown in FIG. 08 is the low circa 0.5 mm amount of measured variation versus the true distance.

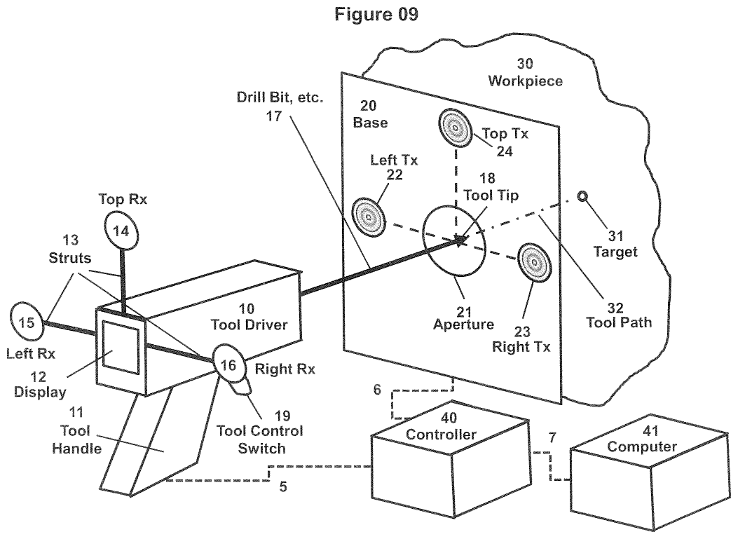

FIG. 09 shows an alternate embodiment of FIG. 01 where the receiving transducers are mounted towards the rear of the tool driver handle in order to permit a lower profile proximal end of the tool, thus allowing working in areas with tighter space constraints. In this (preferred) embodiment, the accuracy of the ultrasonic TOF sensing can include a closer working distance due to providing a longer TOF afforded by the axial distance from moving the transducers from the front to the back.

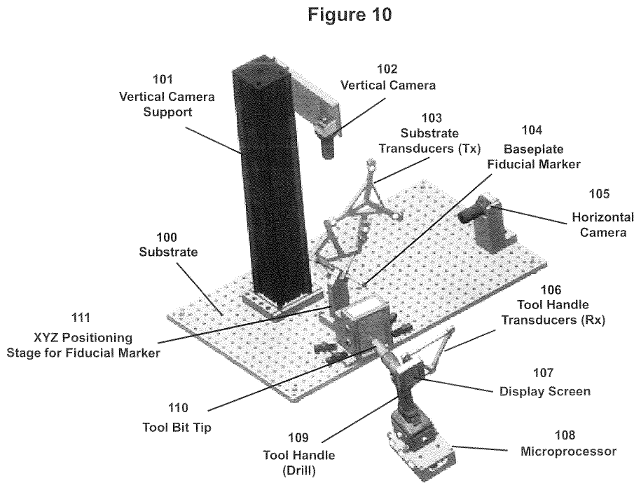

FIG. 10 shows a 3D perspective view of the present invention in a prototype testing platform for the laboratory. Here, a substrate or base plate 100 that mechanically supports all associated fixed items such as the vertical camera support 101 and its associated vertical camera 102, the substrate Tx transducers 103, the base plate fiducial marker 104, the horizontal camera 105, and the 3D XYZ positioning translation stage 111 which locates the said base plate fiducial marker 104. Also shown are the hand-held tool handle 109 with its associated receiving tool handle transducers 106 (microphones), tool bit tip 110, display screen 107, and microprocessor 108. The motion of the hand-held tool handle 109 is measured by combination of video images from the two orthogonally placed cameras and by ultrasonic TOF pings from the substrate transducers 103 and the tool handle transducers 106.

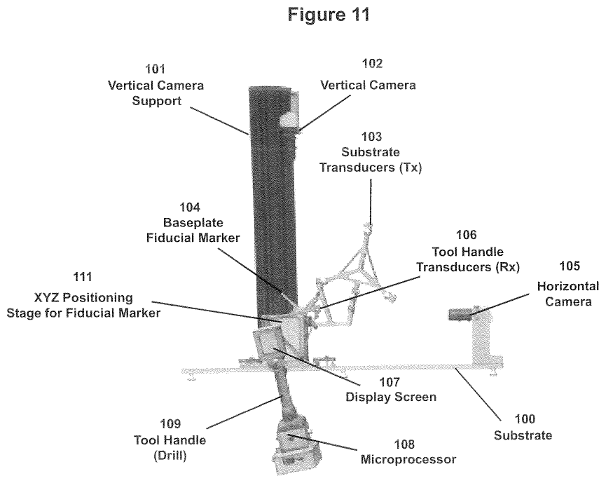

FIG. 11 shows a 3D rendering of the side view of the present invention as shown in FIG. 10 for added clarity. Here, we can see that the tool handle for the drill 109 has a vertically elongated handle for the operator to grasp with the microprocessor located towards the bottom for better ergonomic balance, and the display screen 107 located directly facing the operator for ease of use while operating the tool in a typical hand-held drill forward approach.

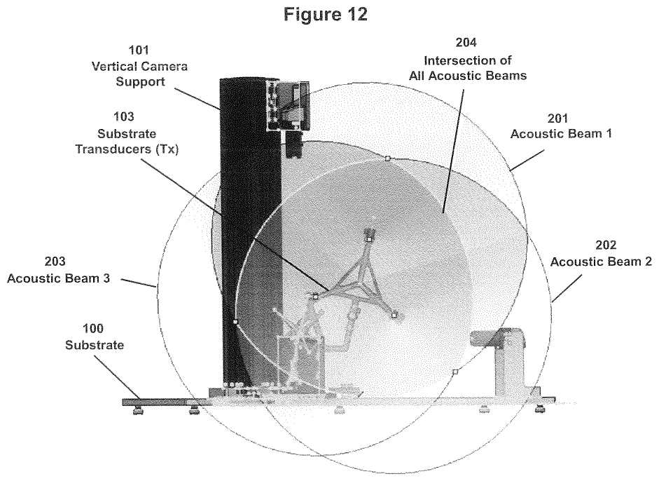

FIG. 12 shows a 3D rendering of the side view of the present invention but with the hand-held tool removed for clarity so that the round cross-sections of the 3 ultrasonic acoustic beams 201, 202, 203 propagating from the substrate transducers 103 can be seen to converge and intersect over a region of intersection of all acoustic beams 204. It is within this intersection of all beams 204 where the ultrasonic TOF distance and attitude determination will work unambiguously.

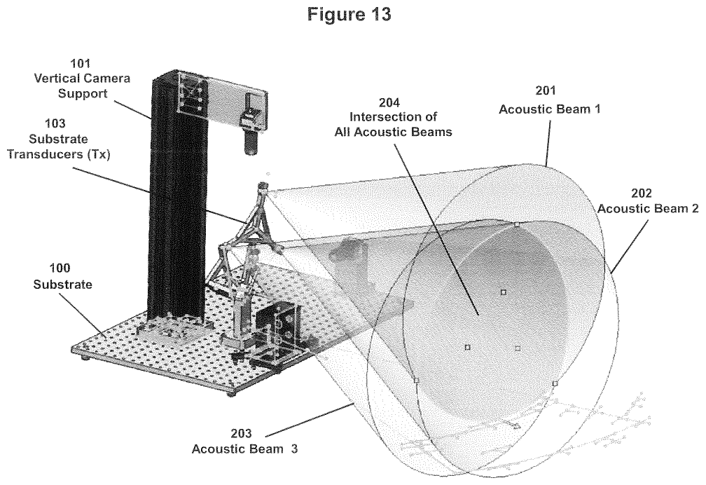

FIG. 13 shows a perspective view of the same system depicted in FIG. 12 such that the sideways view of the 3 acoustic beams 201, 202, 203 can be seen to intersect in a 3D conical space volume 204 along the direction of propagation. The conical volume defined by this 3D “Venn Diagram*” is the active region for this technology to work unambiguously as TOF distance is required from at least 3 transmitters in order to solve the system of 3D equations involved in trilateration.

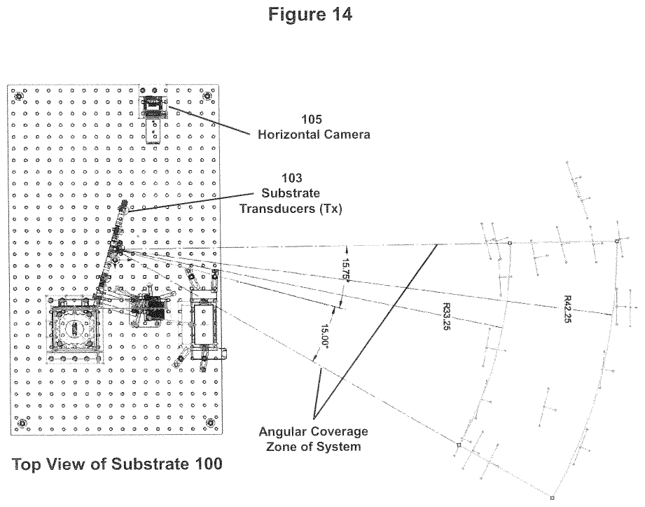

FIG. 14 depicts a line drawing of the Top View of FIG. 13, showing the approximate 30 degree full-angle cone of measurement capability as defined by the projection of the edges of the 3D volume “Venn Diagram” shown in FIG. 13.

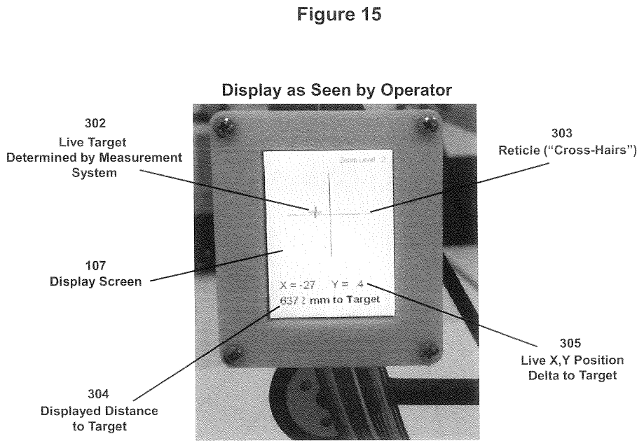

FIG. 15 shows a photograph of the display screen 302 as seen by the operator of the present invention. The 5 DOF data as calculated by the microprocessor using the TOF information from the transducer pings is presented to the operator in an easy-use format that resembles a gun sight reticle, or similarly, a pilot's head-up-display (HUD) used when approaching a landing strip which has both a X, Y, Z linear target requirement as well as a yaw and pitch approach angle requirement. This display makes it easy for the operator to see if the tool path is on target and permits course-corrections if needed in real-time by displaying the cross-hairs 303 relative to a live target 302, while also providing a delta-distance to the target 304 and delta X,Y distances to the target 305. It is also envisaged that the display screen can show other pertinent ancillary data such as drill speed, k-wire extended, etc.

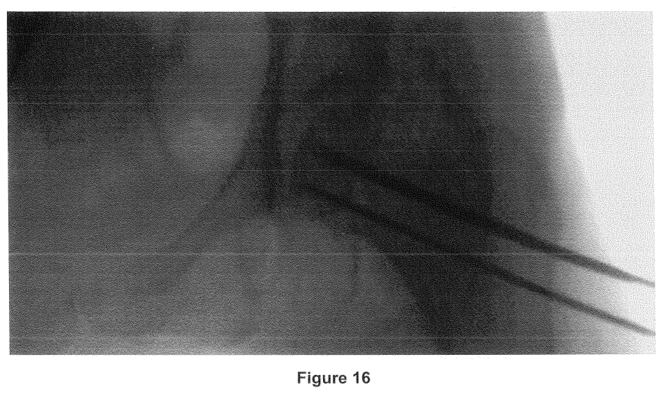

FIG. 16 shows an x-ray of a typical hip joint procedure where a target position is determined by the surgeon through use of the x-ray and the present device would be used for the placement of guide wires as shown in the x-ray.

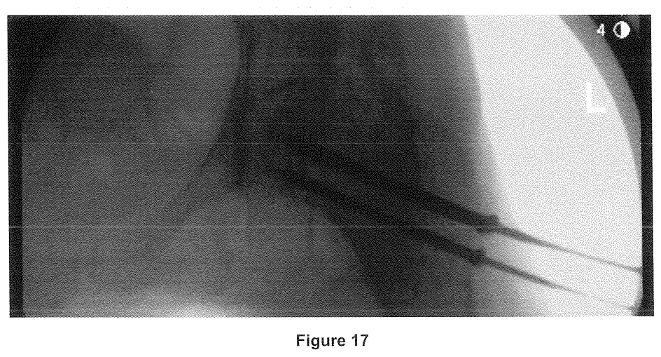

FIG. 17 shows another x-ray of the hip joint procedure with the k-wires in place with the cannulated screws to be inserted over the guide wires.

In a further embodiment of the invention, three fiducials are fixed within the working field to develop a coordinate framework. The surgeon in his/her discretion captures two fluoroscopic images at 90 degrees to each other, and the images are interpreted via machine vision software and displayed on a medical monitor. Then, the surgeon defines the fiducials and optionally, a desired drill start site on the medical monitor in two planes. Next the system registers the target location to the surgical site and the system determines and progressively displays in two dimensions for the selected entry point and the depth, and in real time “so much as possible”, the drill trajectory to a pre-selected target so as to guide the surgeon in drilling from the pre-selected entry point to the pre-selected end point.

The is primarily established in the software portion of the invention similar to a guidance software that allows a user to target the workpath at the correct approach angle. The system display shows the target and also, a box (or alignment bar) around it which shifts up down, and left and right depending on the angular attitude that the user has. Thus, if the box is low relative to the target, the user has to adjust the tool. In accordance with the present invention, the system informs the user of an absolute delta difference and angle of the drill tip vs the target where in a stressful operating room environment, the surgeon needs to simply be given a display to allow him to maneuver the drill to the target with the correct approach angle. Thus, the system guides the user in a translation between 2D and 3D to introduce “course corrections”.

As an example of a medical procedure which utilizes the medical techniques in accordance with the invention, guide wires are placed into the neck of a fractured femoral neck for the subsequent placement of cannulated screws as follows:

The patient is positioned and draped for surgery with the affected hip area placed within the frame of reference of the present invention which includes senders or receivers or combination sender receivers that are coordinated each to an individual channel with sensors on the medical instrument. Using fluoroscopy, orthogonal 2-dimensional x-ray images are taken, and anatomical landmarks are noted on the images with respect to the frame of reference so as to define the coordinate location of the landmarks. Either the system or the surgeon selects a target end point and registers that point on the images (here within the femoral head and opposite an entry point through the femoral neck). The target end point is registered within the frame of reference in order to determine an angle of entry for a guide wire that is placed using the system. A total of three wires will be placed in an inverted triangle for optimal fixation, although it is understood that a single implant or two may be used instead.

On the exterior surface of the patient, the surgeon palpates the bony landmarks for the lateral aspect of the femur opposite to the greater trochanter.

Using a wire driver of the invention, the angle of penetration to line up with the end point is determined by pinging the sensors in the frame of reference with the sensors on scaffolds carried by the wire driver so as to establish a spaced relationship that allows for trilateration between the sensors in the frame of reference and on the instrument. The relationship, including the angle of alignment and the depth of penetration is shown on a display and the system also can include alerts from using other sense forms, such as a vibratory or audio alert if the device strays from the desired alignment. These alerts can use the volume or level of vibration to alert for greater deviations from the desirable alignment.

Following the placement of three guide wires percutaneously through the inferior lateral aspect of the proximal femur within and along the femoral neck to the subchondral aspect of the femoral head (which are each checked for proper placement), cannulated screws are placed and seated over the guide wires to, but not beyond the femoral head in the acetabulum. The placement of the screws is checked, and the guide wires are removed. The wounds are minimal stab wounds which can be closed using butterflies or surgical adhesive or tape.

28. A surgical targeting system as set forth in 27, the offset is based on one or more of the wave number, the microphone displacement, and the transmitter or receiver foci.

29. A three-dimensional aiming system to determine an angle of incline of a terminal workpiece for a surgical tool along a work path in a patient's body from a stat point to a determined end point and comprising: a frame of reference which includes a plurality of senders that define 3D locations and which can be used to establish a set of coordinates for the work path, a plurality of sensors carried by the tool and in communication with the senders, a CPU having machine readable code to determine an alignment of the terminal workpiece relative to the set of coordinates, and a display that informs the user as to the alignment.

30. A three-dimensional aiming system as set forth in 29, wherein the senders and sensors are acoustic or light sensors.

A three-dimensional aiming system as set forth in 30, further including means to calculate a time of flight determination between the senders and sensors.

32. A three-dimensional aiming system as set forth in 29, wherein the senders generate ultrasonic sound pulses with a carrier frequency greater than 20 kHz for use in aiming the terminal workpiece.

33. A three-dimensional aiming system as set forth in 32, wherein the senders generate an acoustic signal which is received by the acoustic receivers and a Fast Fourier Transform (FFT) is used by the CPU to extract phase information from the received acoustic signal in order to derive the time of flight.

34. A three-dimensional aiming system as set forth in 32, wherein the senders are acoustic transmitters that generate an acoustic pulse signal which is received by the sensors which are acoustic receivers which receive a received pulse signal and a discrete Cross Correlation Function (CCF) between the transmitted acoustic pulse signal and the received pulse signal is used by the CPU to derive the time of flight.

A three-dimensional aiming system as set forth in 31, wherein digital signal processing (DSP) is used by the CPU to perform calculations to derive the time of flight.

36. A three-dimensional aiming system as set forth in 31, wherein the senders generate both a carrier at a carder frequency and an acoustic pulse signal which is received by the sensors as a received pulse signal and wherein digital coding schemes are used to modulate the carrier frequency in order to increase a contrast and signal to noise ratios to improve an accuracy of a derivation of the time of flight.

37. A three-dimensional aiming system as set forth in 36, wherein the digital coding scheme is an AA55 code.

38. A three-dimensional aiming system as set forth in 37, wherein an Octave code scheme having a routine is used to automatically extract a phase reversal or an inflection point for the derivation of the time of flight.

39. A three-dimensional aiming system as set forth in 29, further including a video camera to create video images and where the video images are also used simultaneously to aim the terminal workpiece.

A three-dimensional aiming system as set forth in 29, including the further use of fluoroscopic imaging to derive the tool work path determined end point.

A three-dimensional aiming system for positional determination and guidance system as set forth in 29, wherein the accuracy of the guidance of the workpath to the determined end point is within at least +/−1 mm.

42. A three-dimensional aiming system as set forth in 29, wherein the accuracy of the alignment of the terminal work piece is within at least +/−2 degrees to the set of coordinates.

A hand-held orthopedic drill or wire driver having a three-dimensional aiming system to determine an angle of incline of a terminal workpiece for a surgical tool along a work path in a patient's body from a start point to a determined end point and comprising: a frame of reference which includes a plurality of senders which can be used to establish a set of coordinates for the work path, a plurality of sensors carried by the tool and in communication with the senders, a CPU having machine readable code to determine an alignment of the terminal workpiece relative to the set of coordinates, and a display that informs the user as to the alignment.

44. A hand-held orthopedic drill or wire driver as set forth in 43, wherein the senders and sensors are acoustic or light sensors.

45. A hand-held orthopedic drill or wire driver as set forth in 4, further including means to calculate a time of flight determination between the senders and sensors.

A hand-held orthopedic drill or wire driver as set forth in 43, wherein the senders generate ultrasonic sound pulses with a carrier frequency greater than 20 kHz for use in aiming the terminal workpiece.

47. A hand-held orthopedic drill or wire driver as set forth in 46, wherein the senders generate an acoustic signal which is received by the acoustic receivers and a Fast Fourier Transform (FFT) is used by the CPU to extract phase information from the received acoustic signal in order to derive the time of flight.

48. A hand-held orthopedic drill or wire driver as set forth in 46, wherein the senders are acoustic transmitters that generate an acoustic pulse signal which is received by the sensors which are acoustic receivers which receive a received pulse signal and a discrete Cross Correlation Function (CCF) between the transmitted acoustic pulse signal and the received pulse signal is used by the CPU to derive the time of flight.

49. A hand-held orthopedic drill or wire driver as set forth in 45, wherein digital signal processing (DSP) is used by the CPU to perform calculations to derive the time of flight.

50. A hand-held orthopedic drill or wire driver as set forth in 45, wherein the senders generate both a carrier at a carrier frequency and an acoustic pulse signal which is received by the sensors as a received pulse signal and wherein digital coding schemes are used to modulate the carrier frequency in order to increase a contrast and signal to noise ratios to improve an accuracy of a derivation of the time of flight.

A hand-held orthopedic drill or wire driver as set forth in 50, wherein the digital coding scheme is an AA55 code.

52. A hand-held orthopedic drill or wire driver asset forth in 51, wherein an Octave code scheme having a routine is used to automatically extract a phase reversal or an inflection point for the derivation of the time of flight.

53. A hand-held orthopedic drill or wire driver as set forth in 43, further including a video camera to create video images and where the video images are also used simultaneously to aim the terminal workpiece.

54. A hand-held orthopedic drill or wire driver as set forth in 43, including the further use of fluoroscopic imaging to derive the tool work path determined end point.

55. A hand-held orthopedic drill or wire driver as set forth in 43, wherein the accuracy of the guidance of the workpath to the determined end point is within at least +/−1 mm.

56. A hand-held orthopedic drill or wire driver as set forth in 43, wherein the accuracy of the alignment of the terminal work piece is within at least +/−2 degrees to the set of coordinates.

57. A hand-held orthopedic drill or wire driver having a three-dimensional aiming system to determine an angle of incline of a terminal workpiece for a surgical tool along a work path in a patient's body from an initial start point to a determined end point and comprising: a frame of reference which includes a plurality of senders that define two orthogonal planes which can be used to establish a set of coordinates for the work path, a plurality of sensors carried by the tool and in communication with the senders, a CPU having machine readable code to determine an alignment of the terminal workpiece relative to the set of coordinates, and a haptic feedback member that informs the user as to the alignment.

58. A hand-held orthopedic drill or wire driver as set forth in 57, wherein the senders and sensors are acoustic or light sensors.

59. A hand-held orthopedic drill or wire driver as set forth in 58, further including means to calculate a time of flight determination between the senders and sensors.

60. A hand-held orthopedic drill or wire driver as set forth in 57, wherein the senders generate ultrasonic sound pulses with a carrier frequency greater than 20 kHz for use in aiming the terminal workpiece.

A hand-held orthopedic drill or wire driver as set forth in 59, wherein the senders generate an acoustic signal which is received by the acoustic receivers and a Fast Fourier Transform (FFT) is used by the CPU to extract phase information from the received acoustic signal in order to derive the time of flight.

62. A hand-held orthopedic drill or wire driver as set forth in 59, wherein the senders are acoustic transmitters that generate an acoustic pulse signal which is received by the sensors which are acoustic receivers which receive a received pulse signal and a discrete Cross Correlation Function (CCF) between the transmitted acoustic pulse signal and the received pulse signal is used by the CPU to derive the time of flight.

63. A hand-held orthopedic drill or wire driver as set forth in 57, wherein digital signal processing (DSP) is used by the CPU to perform calculations to derive the time of flight.

64. A hand-held orthopedic drill or wire driver as set forth in 57, wherein the senders generate both a carrier at a carrier frequency and an acoustic pulse signal which is received by the sensors as a received pulse signal and wherein digital coding schemes are used to modulate the carrier frequency in order to increase a contrast and signal to noise ratios to improve an accuracy of a derivation of the time of flight.

65. A hand-held orthopedic drill or wire driver as set forth in 64, wherein the digital coding scheme is an AA55 code.

66. A hand-held orthopedic drill or wire driver as set forth in 64, wherein an Octave code scheme having a routine is used to automatically extract a phase reversal or an inflection point for the derivation of the time of flight.

67. A hand-held orthopedic drill or wire driver as set forth in 57, further including a video camera to create video images and where the video images are also used simultaneously to aim the terminal workpiece.

68. A hand-held orthopedic drill or wire driver as set forth in 57, including the further use of fluoroscopic imaging to derive the tool work path determined end point.

69. A hand-held orthopedic drill or wire driver as set forth in 57, wherein the accuracy of the guidance of the workpath to the determined end point is within at least +/−1 mm.

70. A hand-held orthopedic drill or wire driver as set forth in 57, wherein the accuracy of the alignment of the terminal work piece is within at least +/−2 degrees to the set of coordinates.

A hand-held orthopedic drill or wire driver as set forth in 1, that uses a combination of acoustic communication and electrical communication between the hand-held tool and a reference frame.

A hand-held orthopedic drill or wire driver system as set forth in 71, including means to calculate a time of flight determination between the acoustic transmitters and the acoustic receivers.

73. A hand-held orthopedic drill or wire driver as set forth in 71, wherein the acoustic transmitters generate ultrasonic sound pulses with a carrier frequency greater than 20 kHz for use in the guidance of the terminal workpiece.

74. A hand-held orthopedic drill or wire driver as set forth in 73, wherein the acoustic transmitters generate an acoustic signal which is received by the acoustic receivers and a Fast Fourier Transform (FFT) is used by the CPU to extract phase information from the received acoustic signal in order to derive the time of flight.

75. A hand-held orthopedic drill or wire driver as set forth in 73, wherein the acoustic transmitters generate an acoustic pulse signal which is received by the acoustic receivers as a received pulse signal and a discrete Cross Correlation Function (CCF) between the transmitted acoustic pulse signal and the received pulse signal is used by the CPU to derive the time of flight.

76. A hand-held orthopedic drill or wire driver as set forth in 73, wherein digital signal processing (DSP) is used by the CPU to perform calculations to derive the time of flight.

77. A hand-held orthopedic drill or wire driver as set forth in 73, wherein the acoustic transmitters generate both a carrier at a carrier frequency and an acoustic pulse signal which is received by the acoustic receivers as a received pulse signal and wherein digital coding schemes are used to modulate the carrier frequency in order to increase a contrast and signal to noise ratios to improve an accuracy of a derivation of the time of flight.

78. A positional determination and guidance system as set forth in 77, wherein the digital coding scheme is an AA55 code.

79. A hand-held orthopedic drill or wire driver as set forth in 78, wherein a code scheme having a routine is used to automatically extract a phase reversal or an inflection point for the derivation of the time of flight.

80. A hand-held orthopedic drill or wire driver for a surgical tool path determination as described in 57, further including video cameras to create video images and where the video images are also used simultaneously in the guidance of the terminal workpiece along the work path.

A hand-held orthopedic drill or wire driver as set forth in 57, including the further use of fluoroscopic imaging to derive the tool work path determined end point.

82. A hand-held orthopedic drill or wire driver as set forth in 57, wherein the accuracy of the guidance of the workpath to the determined end point is within at least +/−1 mm.

83. A hand-held orthopedic drill or wire driver as set forth in 57, wherein the accuracy of the guidance of the workpath to the determined end point is within at least +/−2 degrees.

84. A method of performing a surgery, comprising the steps of: locating and securing an anatomical area within a three-dimensional reference frame capable of establishing a coordinate system, using an imaging system to define an endpoint spaced from a starting point within the anatomical area and linking the endpoint to the coordinate system to form a set of desired alignment coordinates; providing a CPU having machine readable code and an instrument having a workpiece, and which bears a sender or receiver which are in communication with a corresponding sender or receiver operable with respect to the reference frame and with the CPU to determine a position of the workpiece in the reference frame; aligning instrument by hand in the reference frame such that the alignment of the workpiece corresponds to the desired alignment coordinates.

85. A method of performing a surgery comprising the steps of locating and securing an anatomical area within a three-dimensional reference frame1 providing a CPU having machine readable code and an instrument having a workpiece, and which bears a sender or receiver which are in communication with a corresponding sender or receiver operable with respect to the reference frame and with the CPU to determine a position of the workpiece in the reference frame; aligning instrument progressively and over time by hand in the reference frame such that the alignment of the workpiece corresponds to the desired alignment coordinates.

A method of training or performing a surgery by a user of hand-held instrument and comprising the steps of: locating and securing an anatomical area within a three-dimensional reference frame capable of establishing a coordinate system, using an imaging system to define an endpoint spaced from a starting point within the anatomical area and linking the endpoint to the coordinate system to form a set of desired alignment coordinates; providing a CPU having machine readable code and the hand-held instrument having a workpiece, and which bears a sender or receiver which are in communication with a corresponding sender or receiver operable with respect to the reference frame and with the CPU to determine a position of the workpiece in the reference frame; aligning instrument by the hand in the reference frame such that the alignment of the workplace corresponds to the desired alignment coordinates; and alerting the user as to the location of the Instrument relative to the desired alignment coordinates.

87. A method of performing a hip fixation surgery comprising the steps of locating a hip within a three-dimensional reference frame capable of establishing a coordinate system, using an imaging system to define an endpoint in a femoral head of the hip spaced from a starting point on the proximal femur within the hip and linking the endpoint to the coordinate system to form a set of desired alignment coordinates; providing a CPU having machine readable code and a wire driver having a guide wire, and which bears a sender or receiver which are in communication with a corresponding sender or receiver operable with respect to the reference frame and with the CPU to determine a position of the guide wire in the reference frame; aligning the wire driver in the reference frame such that the alignment of the wire corresponds to the desired alignment coordinates; driving the wire using the wire driver; seating a cannulated screw over the wire driver.

Although the present invention has been described based upon the above embodiments and the data produced by measurement of the performance of the resulting invention that has been reduced to practice, it is apparent to those skilled in the art that certain modifications, variations, and alternative constructions would be apparent, while remaining within the spirit and scope of the invention. In order to determine the metes and bounds of the invention, reference should be made to the following claims.

What is claimed is:

1. A positional determination and guidance system to guide the user of a surgical tool having a terminal workpiece over time along a work path in a patient's body from a start point to a determined target end point and comprising:
a base member which supports a plurality of acoustic transmitters in a known spaced relationship relative to the determined target end point or the start point,
a hand-held tool having a plurality of acoustic receivers in acoustic communication with the acoustic transmitters,
a CPU having machine readable code to determine the progress of the terminal workpiece along the work path toward the determined target end point,
means to calculate a time of flight determination between the acoustic transmitters and the acoustic receivers, and
a display that informs the user as to the guidance of the terminal workpiece along the work path and that allows a user to derive the tool work path determined end point using of fluoroscopic imaging.

2. A positional determination and guidance system as set forth in claim 1, that uses a combination of acoustic communication and electrical communication via hard wires between the hand-held tool and the base member.

3. A positional determination and guidance system as set forth in claim 1, wherein the acoustic transmitters generate ultrasonic sound pulses with a carrier frequency greater than 20 kHz and less than 100 kHz for use in the guidance of the terminal workpiece.

4. A positional determination and guidance system as set forth in claim 1, wherein the acoustic transmitters generate an acoustic pulse signal which is received by the acoustic receivers as a received pulse signal and a discrete Cross Correlation Function (CCF) between the transmitted acoustic pulse signal and the received pulse signal is used by the CPU to derive the time of flight.

5. A positional determination and guidance system as set forth in claim 1, wherein the acoustic transmitters generate an acoustic signal which is received by the acoustic receivers and a Fast Fourier Transform (FFT) is used by the CPU to extract phase information from the received acoustic signal in order to derive the time of flight.

6. A positional determination and guidance system as set forth in claim 1, wherein digital signal processing (DSP) is used by the CPU to perform calculations to derive the time of flight.

7. A positional determination and guidance system as set forth in claim 1, wherein the acoustic transmitters generate both a carrier at a carrier frequency and an acoustic pulse signal which is received by the acoustic receivers as a received pulse signal and wherein an digital coding scheme is used to modulate the carrier frequency in order to increase an contrast and signal to noise ratio to improve an accuracy of a derivation of the time of flight.

8. A positional determination and guidance system as set forth in claim 7, wherein the digital coding scheme is an AA55 code.

9. A positional determination and guidance system as set forth in claim 8, wherein a code scheme having a routine is used to automatically extract a phase reversal or an inflection point for the derivation of the time of flight.

10. A positional determination and guidance system for a surgical tool path determination as described in claim 1 further including video cameras to create video images and where the video images are also used simultaneously in the guidance of the terminal workpiece along the work path.

11. (canceled)

12. A positional determination and guidance system as set forth in claim 1, wherein the accuracy of the guidance of the workpath to the determined end point is at least within +/−1 mm.

13. A positional determination and guidance system as set forth in claim 1, wherein the accuracy of the guidance of the workpath to the determined end point is at least +/−2 degrees.

14. A surgical instrument system which is used by a user holding a surgical instrument having a workpiece which moves along a work path from a starting point to a desired end point in a patient body in a working field comprising:
a plurality of fiducials fixed within the working field to develop a coordinate framework and means to define locations on the work path;
machine vision software loaded in a machine which interprets the locations on the work path and displays them on a medical monitor, and means for the user to define on the medical monitor in two planes a location for at least two of the fiducials and the target end point;
where the instrument system includes the means to register the target end point to the surgical site; and
the system determines and progressively displays in real time, in two dimensions for a determined end point, a work path to the determined end point so as to guide a user holding the surgical instrument in forming the work path to the determined end point wherein the end point is captured using fluoroscopy.

15. A surgical guidance system comprising a surgical drill including ultrasonic senders, a reference frame including receivers, a CPU having hardware including machine readable code to determine TOF of a signal generated by the ultrasonic sender as received by the ultrasonic receiver, and a visual display or feed-back system to inform the surgeon as to how to create a drill pathway through a reference frame which contains the subject patient body part.

16. A surgical system as set forth in claim 15, wherein the reference points for a pathway created by the surgical drill are obtained through digital images.

17. (canceled)

18. A surgical targeting system guided by ultrasonic sender/receiver pairs strategically mounted on a hand-held or potentially robotic drill, the sender/receiver pairs being in proximity to x-ray opaque fiducials positioned relative to a subject surgical area located within a defined three-dimensional reference frame, and a CPU hardware and software to determine the proximity in space of the associated ultrasonic sender/receivers as they change course over time.

19. A surgical targeting system as set forth in claim 18 wherein a surgical pathway is determined by a user and includes a drill entry point and an end point and where the drill entry point and the end point are selected by the user and entered into a computer.

20. A surgical targeting system as set forth in claim 19, wherein the receivers are a wideband microphone.

21. A surgical targeting system as set forth in claim 20, wherein the ultrasonic sender is a piezoelectric ultrasound sender.

22. A surgical targeting system as set forth in claim 18, wherein the sender is used in a pattern that is from 3 to 60° pulses followed by the same number of 180° pulses.

23. A surgical targeting system as set forth in claim 23, wherein the sender is used in a pattern that is from four 0° pulses followed by four 180° pulses.

24. A surgical targeting system as set forth in claim 19, wherein algorithmic means are used to locate the phase inflection point.

25. A surgical targeting system as set forth in claim 24, wherein code is used along with analog data taken to produce a final measurement output.

26. A surgical targeting system as set forth in claim 18, wherein an offset is used to account for detection of wave inversion.

27. A surgical targeting system as set forth in claim 26, the offset is based on one or more of the wave number, the microphone displacement, and the transmitter or receiver foci.

28-87. (canceled)
